(19) United States
(12) Patent Application Publication
(10) Pub. No.: US 2019/0029588 A1
(43) Pub. Date: Jan. 31, 2019

(54) DEVICE, SYSTEM AND METHOD FOR DETECTION AND MONITORING OF DYSPHAGIA OF A SUBJECT

(71) Applicant: KONINKLIJKE PHILIPS N.V., Eindhoven (NL)

(72) Inventor: Mirela Alina WEFFERS-ALBU, Boukoul (NL)

(73) Assignee: KONINKLIJKE PHILIPS N.V., Eindhoven (NL)

(21) Appl. No.: 16/074,568

(22) PCT Filed: Feb. 16, 2017
(86) PCT No.: PCT/EP2017/053552

§ 371 Date: Aug. 1, 2018

(30) Foreign Application Priority Data
Feb. 18, 2016 (EP) 16156302.8

(51) Int. Cl. A61B 5/00; A61B 5/08; A61B 5/0488; A61B 5/11

(52) U.S. Cl. CPC A61B 5/4205; A61B 5/0823; A61B 5/0488; A61B 2562/0219; A61B 5/682; A61B 2562/0204; A61B 5/11

(57) ABSTRACT

The present invention relates to a device, system and method for automatic detection and progression monitoring of dysphagia of a subject, in particular after stroke. The device comprises a signal input for obtaining a mastication signal indicating mastication of the subject, an audio signal representing sounds generated by the subject and a swallowing signal indicating swallowing of the subject, a signal processor for detecting mastication abnormalities from said mastication signal, mastication-to-swallow abnormalities from said mastication signal and said swallowing signal, and aspiration episodes from said audio signal and said swallowing signal, a dysphagia detector for detecting onset and/or progression of dysphagia from detected mastication abnormalities, mastication-to-swallow abnormalities and aspiration episodes and for generating a dysphagia signal indicating information regarding dysphagia of the subject, and a signal output for outputting said dysphagia signal.

### FIELD OF THE INVENTION

The present invention relates to a device, system and method for detection and monitoring of dysphagia of a subject.

### BACKGROUND OF THE INVENTION

Dysphagia (swallowing impairment) is a common side-effect of stroke events due to the damage on the part of the brain that controls the reflex ability to co-ordinate the nerves and muscles involved in the swallowing activity. At least 40% of stroke survivors experience dysphagia. Dysphagia symptoms include difficulty and pain during eating, chewing, drinking or swallowing, as well as coughing due to inhaling food and liquids.

There are several reasons why dysphagia after stroke needs to be managed very closely:

1. Patients with dysphagia very often experience aspiration—that is food, liquids enter the air pathways to lungs. Aside the immediate air pathway blockage hazard, food or liquid particles inhaled during aspiration often lead to pneumonia. Given the weak state of stroke patients, suffering from pneumonia in addition can literally lead to death.
2. Another common side-effect of dysphagia is malnutrition as well as dehydration—which delay stroke recovery, and are extremely critical especially in elderly patients already weakened by the stroke event.

In that sense dysphagia onset needs to be determined as soon as possible after stroke in order to ensure safety measures. At the same time dysphagia progression needs to be monitored to adjust feeding and drinking (e.g. frequency, consistency of food and liquids, etc.) according to patients' condition and needs.

The current practice involves immediate screening and assessment after the stroke event as well as further investigation tests, such as videofluoroscopy, which is a procedure that involves taking a series of x-ray images of the parts of your body involved in swallowing, and fibre-optic (or optical fiber) endoscopic evaluation, which involves a long thin, flexible tube that has a light source and a camera on the end of the endoscope that is placed up one nostril and moved into position so that it can view images of the back of the patient throat and the structures involved in swallowing.

The current practice has the disadvantages that the aforementioned tests (videofluoroscopy, fibre-optic (or optical fiber) endoscopic evaluation) can be done only once following the stroke event. Further, patients are not monitored at home to adjust their feeding and hydration support/prescription, thereby making the known tests impractical for patient monitoring and use at home. There is, however, a need to continue monitoring stroke patients and to adjust their food and liquid intake (e.g. frequency, consistency of food and liquids, etc.) according to the progression of their dysphagia condition.

### SUMMARY OF THE INVENTION

It is an object of the present invention to provide a device, system and method that enable automatic detection and progression monitoring of dysphagia of a subject, in particular after stroke.

In a first aspect of the present invention, a device for detection and monitoring of dysphagia of a subject is presented, said device comprising:
a signal input for obtaining a mastication signal indicating mastication of the subject, an audio signal representing sounds generated by the subject and a swallowing signal indicating swallowing of the subject,
a signal processor for detecting mastication abnormalities from said mastication signal, mastication-to-swallow abnormalities from said mastication signal and said swallowing signal, and aspiration episodes from said audio signal and said swallowing signal,
a dysphagia detector for detecting onset and/or progression of dysphagia from detected mastication abnormalities, mastication-to-swallow abnormalities and aspiration episodes and for generating a dysphagia signal indicating information regarding dysphagia of the subject, and
a signal output for outputting said dysphagia signal.

In a further aspect of the present invention, a system for detection and monitoring of dysphagia of a subject is presented, said system comprising:
a mastication signal sensor for acquiring a mastication signal indicating mastication of the subject,
an audio signal sensor for acquiring an audio signal representing sounds generated by the subject,
a swallowing signal sensor for acquiring a swallowing signal indicating swallowing of the subject, and
a device as disclosed herein for detection and monitoring of dysphagia of the subject from the acquired mastication signal, swallowing signal and audio signal.

In yet further aspects of the present invention, there are provided a corresponding method and a computer program which comprises program code means for causing a computer to perform the steps of the method disclosed herein when said computer program is carried out on a computer as well as a non-transitory computer-readable recording medium that stores therein a computer program product, which, when executed by a processor, causes the method disclosed herein to be performed.

Preferred embodiments of the invention are defined in the dependent claims. It shall be understood that the claimed method, system, computer program and medium have similar and/or identical preferred embodiments as the claimed device and as defined in the dependent claims.

The present invention is based on the idea to provide means which enable to determine early on the onset of dysphagia as well as to monitor its progression after patient discharge, in the context of the home, which the current state of art cannot provide. For this purpose a mastication signal indicating mastication of the subject, an audio signal representing sounds generated by the subject and a swallowing signal indicating swallowing of the subject are acquired by corresponding sensors. These signals are processed into mastication abnormalities, mastication-to-swallow abnormalities and aspiration episodes. It has been found that this information allows detecting onset and/or progression of dysphagia, which are then represented by a dysphagia signal that may be outputted, e.g. to a caregiver, a doctor, a nurse and/or the user.

Hereby, the detection of aspiration episodes provides advantages because inhaling liquid or food particles may lead to either asphyxiation and death onset as an immediate result of the aspiration event or resulting pulmonary infection and death by sepsis, due to particles of food having remained blocked in the lungs tissues. Also for these reasons it is important to monitor dysphagia since a patient may not only become malnourished but may die from aspiration episodes.

In an embodiment said signal processor is configured to detect mastication abnormalities from said mastication signal and said audio signal in order to distinguish mastication activity from speaking activity. This improves the detection of dysphagia and avoids misdetections.

In an embodiment said signal processor is configured to detect abnormalities regarding mastication duration as mastication abnormalities, in particular by comparing a detected mastication duration with a mastication duration threshold. This further improves the detection of dysphagia. The mastication duration threshold may be predetermined, e.g. from measurements with a plurality of persons, from generally accepted medical guidelines or with the person under examination so that for each person an individual mastication duration threshold is used.

Further, in an embodiment said signal processor is configured to detect abnormalities regarding mastication-to-swallow duration from the beginning of a mastication activity to a swallow event as mastication-to-swallow abnormalities, in particular by comparing a detected mastication-to-swallow duration with a mastication-to-swallow threshold. This also further improves the detection of dysphagia. The mastication-to-swallow threshold may also be predetermined, e.g. from measurements with a plurality of persons, from generally accepted medical guidelines or with the person under examination so that for each person an individual mastication-to-swallow threshold is used.

The signal processor may further be configured to detect the aspiration episode by detecting if a swallow event is followed by one or more cough events. A cough event shortly following a swallow event clearly indicates an aspiration episode. A cough event may thereby be detected from said audio signal and/or a movement signal representing movement of the subject's larynx, chest wall or abdominal wall.

The signal processor may also be configured to detect the aspiration episode by detecting a swallow-to-cough duration from a swallow event to a cough event and/or by detecting the number of cough events following a swallow event within a predetermined time period. An aspiration episode may thereby be detected by comparing the detected swallow-to-cough duration to swallow-to-cough duration threshold and/or by comparing the detected number of cough events to a cough events threshold.

According to another embodiment said dysphagia detector is configured to detect onset of dysphagia if a mastication abnormality, a mastication-to-swallow abnormality and/or one or more aspiration episodes are detected and/or to detect as progression of dysphagia the number and/or rate of mastication abnormalities, mastication-to-swallow abnormalities and/or aspiration episodes. Thus, a reliable way of detecting onset and/or progression of dysphagia is possible using the information determined from the sensed data.

Various kinds of sensors may be used for this purpose. Practical embodiments may use a mastication signal sensor that comprises an electromyography sensor and/or a motion sensor for arrangement at the subject's jaw area, an audio signal sensor that comprises a microphone or a plurality of microphones, in particular a contact microphone and/or an acoustic microphone, for arrangement at or close to the subject, and/or a swallowing signal sensor that comprises a laryngeal sensor for arrangement at the subject's throat, in particular in the area of the subject's larynx.

Instead of such sensors which are generally mounted at or close to the subject's body, a camera may be used for acquiring video data of the subject, wherein said mastication signal sensor is configured to acquire said mastication signal from said video data and/or said swallowing signal sensor is configured to acquire said swallowing signal from said video data. Hence, it is generally possible to obtain the desired data without any body mounted (or wearable) sensors.

In still another aspect of the present invention, a device and a corresponding method for detection and monitoring of dysphagia of a subject is presented, said device comprising, i) a signal input for obtaining a mastication signal indicating mastication of the subject, an audio signal representing sounds generated by the subject and a swallowing signal indicating swallowing of the subject, ii) a signal processor for detecting mastication abnormalities from said mastication signal, mastication-to-swallow abnormalities from said mastication signal and said swallowing signal, and aspiration episodes from said audio signal and said swallowing signal, iii) a dysphagia detector for detecting onset and/or progression of dysphagia from detected mastication abnormalities, mastication-to-swallow abnormalities and aspiration episodes and for generating a dysphagia signal indicating information regarding dysphagia of the subject, and iv) a signal output for outputting said dysphagia signal.

### BRIEF DESCRIPTION OF THE DRAWINGS

These and other aspects of the invention will be apparent from and elucidated with reference to the embodiment(s) described hereinafter. In the following drawings

FIG. 1 shows a schematic diagram of a first embodiment of a system and a device according to the present invention,

FIG. 2 shows an illustration of the mounting of various sensors at the subject's head,

FIG. 3 shows a schematic diagram of a second embodiment of a system and a device according to the present invention,

FIG. 4 shows a graph of an exemplary mastication signal,

FIG. 5 shows a graph of an exemplary swallowing signal, and

FIG. 6 shows graphs of an exemplary motion signal and exemplary audio signals.

### DETAILED DESCRIPTION OF THE INVENTION

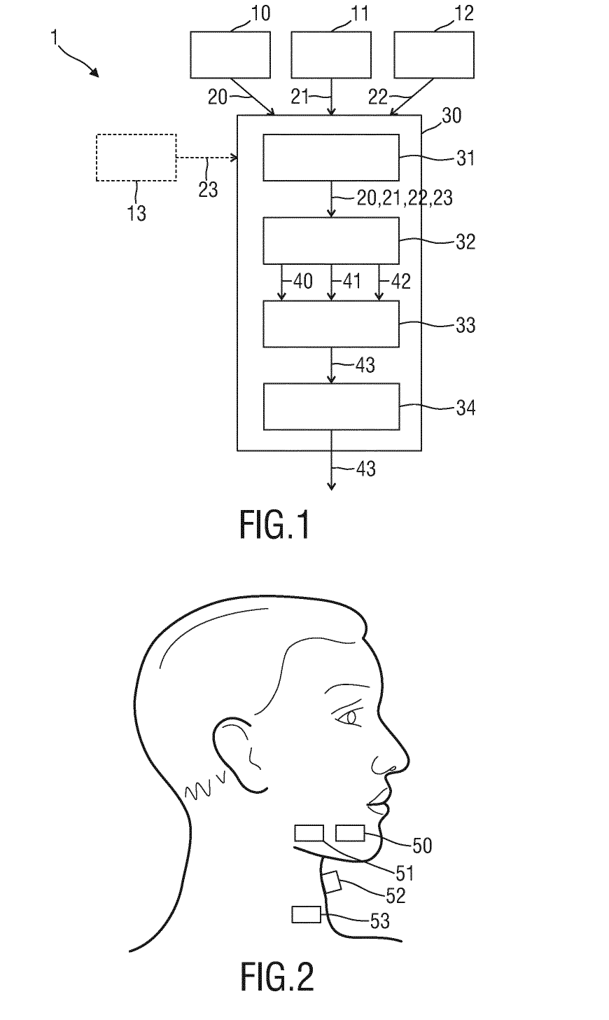

FIG. 1 shows a schematic diagram of a first embodiment of a system 1 and a device 30 according to the present invention for detection and monitoring of dysphagia of a subject, such as a patient in a hospital or in home care. The system 1 comprises various types of sensors including a mastication signal sensor 10 for acquiring a mastication signal 20 indicating mastication of the subject, an audio signal sensor 11 for acquiring an audio signal 21 representing sounds generated by the subject, and a swallowing signal sensor 12 for acquiring a swallowing signal 22 indicating swallowing of the subject. The system 1 further comprises a device 30 for detection and monitoring of dysphagia of the subject from the acquired mastication signal 20, swallowing signal 21 and audio signal 22.

FIG. 2 shows an illustration of an exemplary mounting of an exemplary set of various types of sensors at the subject's head.

The mastication signal sensor 10 may e.g. comprise an electromyography (EMG) sensor 50 and/or a motion sensor 51 (e.g. an accelerometer) that is arranged at the subject's jaw area. For instance, the EMG sensor 50 may be placed on the subject's jaw. Alternatively or additionally, the motion sensor 51 can be used, e.g. placed behind the subject's jaw or under the subject's ear.

The audio signal sensor 11 may comprise a microphone 52, in particular a contact microphone and/or an acoustic microphone, that is arranged at or a close to the subject, e.g. at the subject's throat or cheek.

The swallowing signal sensor 12 may comprise a laryngeal sensor 53 that is arranged at the subject's larynx, more precisely at the subject's throat in the area of the subject's larynx.

The device 30 may be implemented in hard- and/or software, e.g. as application program (“app”) running on a user device, such as a smartphone, tablet, wearable sports activity monitor, laptop, etc., or on a computer or workstation, e.g. in a hospital or care center.

The device 30 comprises a signal input 31, e.g. a data interface, for obtaining, i.e. retrieving (e.g. downloading) or receiving, the above mentioned various signals from the various sensors 10-12 (50-53 in the embodiment shown in FIG. 2). The signals may be obtained wirelessly, e.g. using a communication network such as Bluetooth or WiFi, or in a wired manner by use of cabling. There may also be a central transmitter (not shown) provided at the subject's body that collects the signals from the various sensors and then sends them to the device or provides them for retrieval by the device 30.

The device 30 further comprises a signal processor 32 for detecting mastication abnormalities 40 from said mastication signal 20, mastication-to-swallow abnormalities 41 from said mastication signal 20 and said swallowing signal 22, and aspiration episodes 42 from said audio signal 21 and said swallowing signal 22.

The device 30 further comprises a dysphagia detector 33 for detecting onset and/or progression of dysphagia from detected mastication abnormalities 40, mastication-to-swallow abnormalities 41 and aspiration episodes 42 and for generating a dysphagia signal 43 indicating detected onset and/or progression of dysphagia of the subject. The signal processor 32 and the dysphagia detector 33 may be commonly implemented by a processor or computer.

The device 30 further comprises a signal output 34 for outputting said dysphagia signal 43 indicating information regarding dysphagia of the subject. The signal output 34 may include a display, such as the display of user device or computer, for issuing the determined dysphagia signal 43 directly for information of and/or use by the user. Alternatively or additionally, the signal output may include a transmission unit, such as a WiFi interface, LAN interface or Bluetooth interface, for transmitting the determined dysphagia signal 43 to a third entity, e.g. a remote user device (e.g. a smartphone or tablet) of a doctor, caregiver or nurse, or a central computer or database of a hospital.

In a preferred embodiment the system 1 may further comprise a movement sensor 13, such as an accelerometer, for acquiring a movement signal 23 representing movement of the subject's larynx, chest wall or abdominal wall. For this purpose the movement sensor 13 is preferably mounted at the respective body part of the subject. The movement signal 23 is preferably used in the detection of a cough event, optionally together with the audio signal 21, by the signal processor 32.

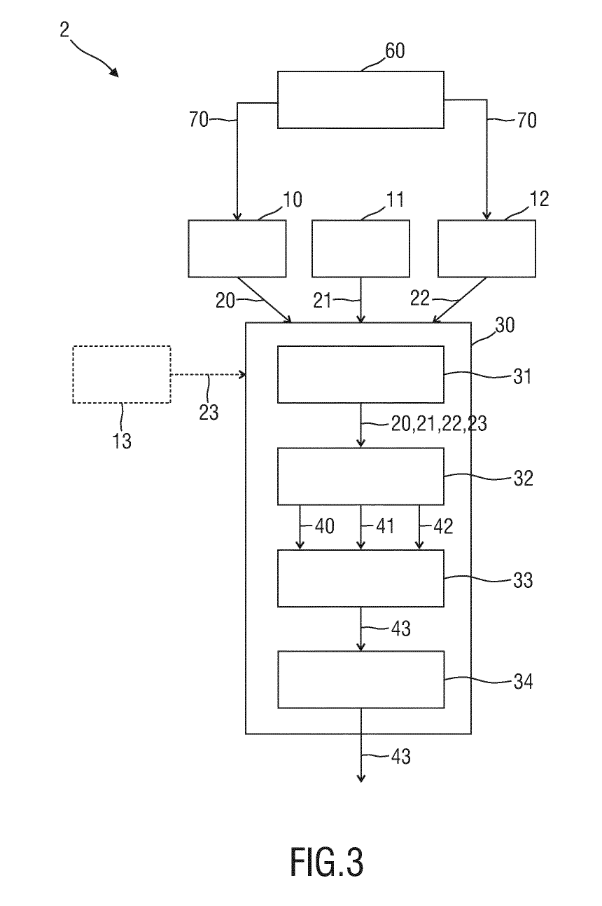

FIG. 3 shows a schematic diagram of a second embodiment of a system 2 and a device 30 according to the present invention. In this embodiment a camera 60 is provided for acquiring video data 70 of the subject. Said video data 70 is used by the mastication signal sensor 10 to acquire said mastication signal 20 and/or by the swallowing signal sensor 12 to acquire said swallowing signal 22. For this purpose image processing means are preferably used to identify mastication and/or swallowing, optionally after segmentation of the respective body part used for said identification. Hence, if the video data 70 are used for obtaining the respective signals 10 and/or 12, a corresponding contact sensor (as e.g. illustrated in FIG. 2) for acquiring the respective signals, may be omitted. In another embodiment both sources for the same signal may be used (e.g. combined through averaging) to increase accuracy and reliability.

Using the above described systems and methods particularly onset of dysphagia can be detected and progression of dysphagia can be monitored. Exemplary implementations and options will be explained in the following:

For detecting onset of dysphagia (e.g. by an onset detection component), the following may be detected by automatic detection:
if the mastication duration (MD) is higher than commonly known thresholds; said interval expresses patient difficulty to processing food;
if the mastication-to-swallow interval (MSI) higher than predetermined (e.g. commonly known) thresholds; said interval expresses patient difficulty to both processing food and activating swallow reflex leading to prolonged mastication activity followed by a delayed swallow event;
of aspiration episodes, which may be done by detecting coughing events before, during and/or after swallowing events and, optionally, detecting hoarse voice.

Onset of dysphagia may thus e.g. be determined by one or more of an abnormally high MD over more than one meal, an imbalanced MSI over more than one meal, and more than one aspiration episode.

For monitoring progression of dysphagia (e.g. by a progression monitoring component), trends over time of MSI index and/or of an incidence rate of aspiration episodes may be monitored. Significant decreases or increases in either or both parameters may be used as indicators for worsening or improvement of the subject's progression of dysphagia.

The system may further provide recommendations (e.g. by an intervention component or the signal output) on how to manage dysphagia by means of personalized interventions regarding frequency, quantity of meals, consistency of food, and consistency of liquids, posturing during eating, chewing & swallowing exercises, support cutlery for eating, special cup for drinking, etc. Further, a closed loop assessment of interventions' effectiveness based on dysphagia monitoring and assessment after interventions may be implemented. Still further, an automatic alarm may be generated and issued, said alarm triggering a caregiver as soon as an ongoing aspiration episode has been detected.

The dysphagia onset detection and dysphagia progression monitoring may use the following automatic functionality:
i) MSI automatic detection based on the components
i1) mastication activity detection;
i2) swallow events detection;
i3) detection of abnormal MD;
i4) detection of imbalanced MSI.
ii) Aspiration episodes detection based on the components:
ii1) cough events detection;
ii2) swallow events detection (like i2); and
ii3) detection of correlation of cough events with swallow events.

Exemplary embodiments for implementing each of the above mentioned functionalities i) and ii) and their components will be explained in the following:

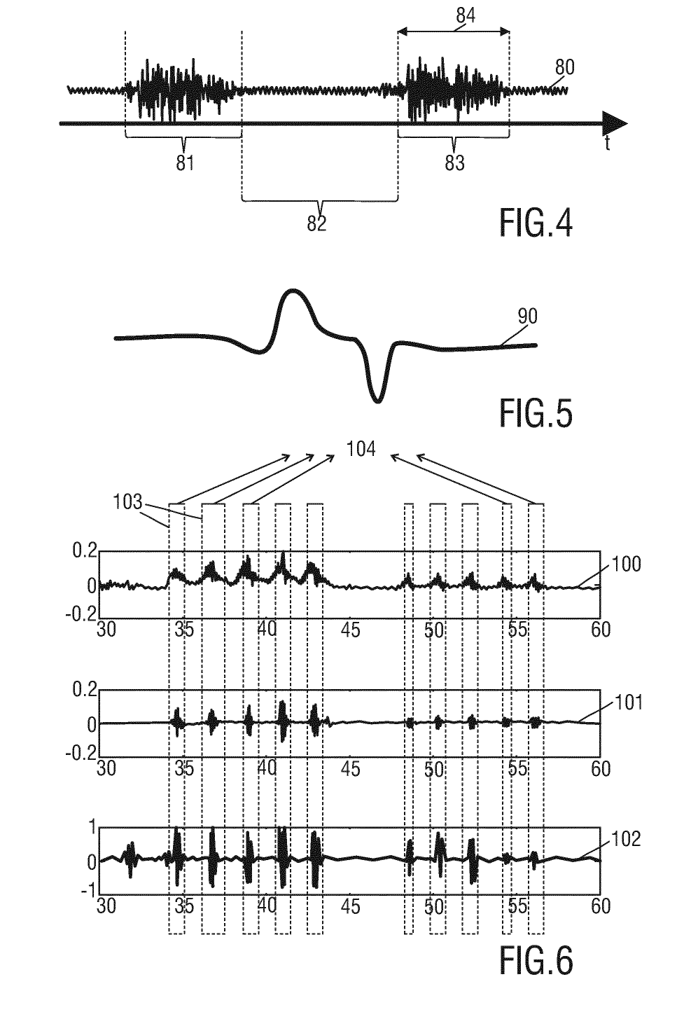

Mastication activity detection (i1) may use an EMG sensor placed on patient jaw (alternatively a motion sensor can be used, placed behind patient jaw or under ear) and/or a miniature microphone. In the signal from the EMG sensor (or the motion sensor), i.e. a mastication signal 80 as shown as an example in FIG. 4, a mastication pattern is recognized as a sequence of peaks in the signal amplitude significantly higher than during previous inactive periods and similar duration. In the mastication signal 80 shown in FIG. 4 mastication activity patterns 81, 83 (having a duration 84) are interspersed with inactive periods 82 (at which the mastication muscles are at rest). As can be seen, the amplitude of the mastication signal 80 during mastication (periods 81, 83) is significantly higher than during inactive periods 82.

To ensure robust differentiation from speaking activity, which involves voice activation as well, the audio signal 22, e.g. from a microphone that recognizes patient voice in foreground sound, may be evaluated.

Swallow events detection (i2; ii2) may use a laryngeal sensor placed on the patient's larynx. A swallowing pattern may be recognized in a swallowing signal 90, as shown as an example in FIG. 5, as succession of high and low peak in the signal amplitude.

Detection of abnormal MD (i3) may be achieved by detecting when the mastication duration consistently significantly exceeds a mastication duration threshold, such as a common threshold (e.g. 5 seconds, or more generally a value in the range of 1-10 seconds, preferably 5-10 seconds).

Detection of imbalanced MSI (i4) may be achieved by detecting when the duration from the beginning of the mastication activity pattern followed by a swallow event consistently significantly exceeds a mastication-to-swallow threshold, such as common threshold (e.g. 5 seconds, or more generally a value in the range of 1-10 seconds, preferably 5-10 seconds).

Cough events detection (ii1) may use one or more of an accelerometer, a contact microphone and an acoustic microphone. An accelerometer signal 100, a contact microphone signal 101 and an acoustic microphone signal 102 are shown in FIG. 6. A coughing event pattern of cough events 104 in periods 103 is clearly present in all three signals 100, 101, 102 as a succession of peaks of significantly higher amplitude than during inactive periods.

Detection of correlation of cough events with swallow events (ii3) may detect if a swallow event revealed in the laryngeal sensor signal is immediately followed by repeated cough events detected in one of the accelerometer/contact/audio microphone sensor signal. This allows detection if an aspiration event is in progression.

In summary, the present invention provides a device, system and method that enable the detection of the onset of dysphagia as well as the monitoring of its progression, e.g. in the context of the home. Further, recommendations for personalized interventions tailored according to the subject (e.g. patient) up to date condition may be generated and issued.

While the invention has been illustrated and described in detail in the drawings and foregoing description, such illustration and description are to be considered illustrative or exemplary and not restrictive; the invention is not limited to the disclosed embodiments. Other variations to the disclosed embodiments can be understood and effected by those skilled in the art in practicing the claimed invention, from a study of the drawings, the disclosure, and the appended claims.

A processor, such as the signal processor, a processing unit, for instance a controller, implements (part of) the control method. A processor can be implemented in numerous ways, with software and/or hardware, to perform the various functions required. The processor may employs one or more microprocessors that may be programmed using software (e.g., microcode) to perform the required functions. Alternatively, the processor may be implemented as a combination of dedicated hardware to perform some functions and/or be coupled to a further processor (e.g., one or more programmed microprocessors and associated circuitry) for performing other functions.

Examples of processor components that may be employed in various embodiments of the present disclosure include, but are not limited to, conventional microprocessors, application specific integrated circuits (ASICs), and field-programmable gate arrays (FPGAs).

In various implementations, the processor be associated with one or more storage media such as volatile and non-volatile computer memory such as RAM, PROM, EPROM, and EEPROM. The storage media may be encoded with one or more programs that, when executed on one or more processors and/or controllers, perform at the required functions. Various storage media may be fixed within the processor or may be transportable, such that the one or more programs stored thereon can be loaded into a processor.

In the claims, the word “comprising” does not exclude other elements or steps, and the indefinite article “a” or “an” does not exclude a plurality. A single element or other unit may fulfill the functions of several items recited in the claims. The mere fact that certain measures are recited in mutually different dependent claims does not indicate that a combination of these measures cannot be used to advantage.

A computer program may be stored/distributed on a suitable non-transitory medium, such as an optical storage medium or a solid-state medium supplied together with or as part of other hardware, but may also be distributed in other forms, such as via the Internet or other wired or wireless telecommunication systems.

Any reference signs in the claims should not be construed as limiting the scope.

1. Device for detection and monitoring of dysphagia of a subject, said device comprising:
a signal input for obtaining a mastication signal indicating mastication of the subject, an audio signal representing sounds generated by the subject and a swallowing signal indicating swallowing of the subject,
a signal processor for detecting mastication abnormalities from said mastication signal, mastication-to-swallow abnormalities from said mastication signal and said swallowing signal, and aspiration episodes from said audio signal and said swallowing signal,
a dysphagia detector for detecting onset and/or progression of dysphagia from detected mastication abnormalities, mastication-to-swallow abnormalities and aspiration episodes and for generating a dysphagia signal indicating information regarding dysphagia of the subject, and
a signal output for outputting said dysphagia signal.

2. Device as claimed in claim 1,
wherein said signal processor is configured to detect an aspiration episode by detecting if a swallow event is followed by one or more cough events.

3. Device as claimed in claim 1,
wherein said signal processor is configured to detect mastication abnormalities from said mastication signal and said audio signal in order to distinguish mastication activity from speaking activity.

4. Device as claimed in claim 1,
wherein said signal processor is configured to detect abnormalities regarding mastication duration as mastication abnormalities.

5. Device as claimed in claim 3,
wherein said signal processor is configured to detect abnormalities regarding mastication duration by comparing a detected mastication duration with a mastication duration threshold.

6. Device as claimed in claim 1,
wherein said signal processor is configured to detect abnormalities regarding mastication-to-swallow duration from the beginning of a mastication activity to a swallow event as mastication-to-swallow abnormalities, in particular by comparing a detected mastication-to-swallow duration with a mastication-to-swallow threshold.

7. Device as claimed in claim 2,
wherein said signal processor is configured to detect a cough event from said audio signal and/or a movement signal representing movement of the subject's larynx, chest wall or abdominal wall.

8. Device as claimed in claim 2,
wherein said signal processor is configured to detect the aspiration episode by detecting a swallow-to-cough duration from a swallow event to a cough event and/or by detecting the number of cough events following a swallow event within a predetermined time period.

9. Device as claimed in claim 8,
wherein said signal processor is configured to detect the aspiration episode by comparing the detected swallow-to-cough duration to a swallow-to-cough duration threshold and/or by comparing the detected number of cough events to a cough events threshold.

10. Device as claimed in claim 1,
wherein said dysphagia detector is configured to detect onset of dysphagia if a mastication abnormality, a mastication-to-swallow abnormality and/or one or more aspiration episodes are detected and/or to detect as progression of dysphagia the number and/or rate of mastication abnormalities, mastication-to-swallow abnormalities and/or aspiration episodes.

11. System for detection and monitoring of dysphagia of a subject, said system comprising:
a mastication signal sensor for acquiring a mastication signal indicating mastication of the subject,
an audio signal sensor for acquiring an audio signal representing sounds generated by the subject,
a swallowing signal sensor for acquiring a swallowing signal indicating swallowing of the subject, and
a device as defined in claim 1, for detection and monitoring of dysphagia of the subject from the acquired mastication signal, swallowing signal and audio signal.

12. System as claimed in claim 11,
wherein said mastication signal sensor comprises an electromyography sensor and/or a motion sensor for arrangement at the subject's jaw area,
wherein said audio signal sensor comprises a microphone, in particular a contact microphone and/or an acoustic microphone, for arrangement at or a close to the subject, and/or
wherein said swallowing signal sensor comprises a laryngeal sensor for arrangement at the subject's throat, in particular in the area of the subject's larynx.

13. System as claimed in claim 11,
further comprising a camera for acquiring video data of the subject,
wherein said mastication signal sensor is configured to acquire said mastication signal from said video data and/or said swallowing signal sensor is configured to acquire said swallowing signal from said video data.

14. Method for detection and monitoring of dysphagia of a subject, said method comprising:
obtaining a mastication signal indicating mastication of the subject, an audio signal representing sounds generated by the subject and a swallowing signal indicating swallowing of the subject,
detecting mastication abnormalities from said mastication signal, mastication-to-swallow abnormalities from said mastication signal and said swallowing signal, and aspiration episodes from said audio signal and said swallowing signal,
detecting onset and/or progression of dysphagia from detected mastication abnormalities, mastication-to-swallow abnormalities and aspiration episodes,
generating a dysphagia signal indicating information regarding dysphagia of the subject, and
outputting said dysphagia signal.

15. Method as claimed in claim 14, further comprising detecting an aspiration episode by detecting if a swallow event is followed by one or more cough events.

16. Method as claimed in claim 14, further comprising detecting mastication abnormalities from said mastication signal and said audio signal in order to distinguish mastication activity from speaking activity.

17. Computer program product comprising a computer readable medium having computer readable code embodied therein, the computer readable code being configured such that, on execution by a suitable computer or processor, the computer or processor is caused to perform the steps of the method of claim 14.
